(19) United States
(12) Patent Application Publication
(10) Pub. No.: US 2013/0172588 A1
(43) Pub. Date: Jul. 4, 2013

(54) CONTINUOUS PHOTOLYTIC PROCESS FOR THE PREPARATION OF VITAMIN D RELATED SUBSTANCES

(71) Applicants: William Edward Bauta, San Antonio, TX (US); Bruce Girard Anderson, San Antonio, TX (US); William R. Cantrell, JR., San Antonio, TX (US)

(72) Inventors: William Edward Bauta, San Antonio, TX (US); Bruce Girard Anderson, San Antonio, TX (US); William R. Cantrell, JR., San Antonio, TX (US)

(73) Assignee: GENZYME CORPORATION, Cambridge, MA (US)

(21) Appl. No.: 13/821,686

(22) PCT Filed: Sep. 1, 2011
(86) PCT No.: PCT/US11/50187

§ 371 Date: Mar. 8, 2013

Related U.S. Application Data
(60) Provisional application No. 61/397,701, filed on Sep. 10, 2010.

(51) Int. Cl. C07C 401/00

(52) U.S. Cl. CPC C07C 401/00
USPC 552/653

(57) ABSTRACT

The present application provides a novel method for generation of a vitamin D2 compound using a continuous flow photoisomerization reactor. A compound represented by formula I: [structure] as further defined herein, is mixed with a solvent and a sensitizer, and is then passed through the continuous flow photoisomerization reactor. If X3 and X4 of formula II is tert-butyldimethylsilyl, then formula II is mixed with a deprotection reagent to obtain the vitamin D2 analog.

### CROSS-REFERENCE TO RELATED APPLICATIONS

This application claims priority to U.S. provisional application No. 61/397,701, filed Sep. 10, 2010, the content of which is incorporated herein by reference in its entirety.

### BACKGROUND

Metabolic hydroxylation at both C(1) and C(25) is required in order to convert vitamin D₂ compounds to their active form, 1α,25-dihydroxyvitamin D₂. Although this conversion is typically accomplished in healthy patients through a combination of renal and hepatic metabolism, the chemical introduction of the C(1) hydroxyl group to a suitable precursor can be a challenging operation. The present invention provides a continuous photolytic process for the preparation of vitamin D₂ analogs.

### SUMMARY

The present application provides methods of making vitamin D₂ compounds with a continuous flow photoisomerization reactor. In one embodiment, the method comprises mixing a compound represented by formula I with a solvent to form a first mixture. The compound of formula I is:

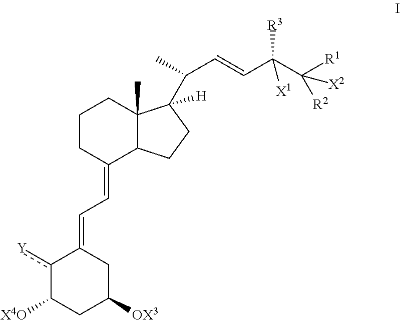

wherein R¹ and R² are identical or different and are hydrogen, hydroxyl, lower alkyl, lower fluoroalkyl, O-lower alkyl, lower alkenyl, lower fluoroalkenyl, O-lower alkenyl, O-lower acyl, O-aromatic acyl, lower cycloalkyl with the proviso that R¹ and R² cannot both be an alkenyl group, or taken together with the carbon to which they are bonded, form a C₃-C₈ cyclocarbon ring; R³ is lower alkyl, lower alkenyl, lower fluoroalkyl, lower fluoroalkenyl, O-lower alkyl, O-lower alkenyl, O-lower acyl, O-aromatic acyl or lower cycloalkyl; X¹ is hydrogen or hydroxyl, or, taken with R³, constitutes a bond when R³ is an alkenyl group, and X² is hydrogen or hydroxyl, or, taken with R¹ or R², constitutes a double bond, and X³ and X⁴ is hydrogen or tert-butyldimethylsilyl; and Y is a methylene group if the bond to Y is a double bond or is a methyl group or hydrogen if the bond to Y is a single bond.

Suitably the solvent can be selected from the group consisting of heptane, methanol, toluene, 1,2-dichloroethane, t-butyl methyl ether, ethyl acetate, and mixtures thereof. Additionally, in some embodiments, the concentration of the compound of formula I in the solvent is about 5 mg/mL to about 50 mg/mL. In other embodiments, the concentration of the compound of formula I in the solvent is at least about 50 mg/mL. In some embodiments the solvent can be deoxygenated by any known deoxygenation technique, such as sparging He.

The first mixture is then combined with a sensitizer to form a second mixture. Suitably, this sensitizer can be 9-acetylanthracene. In some embodiments, the ratio of the sensitizer to the first mixture in step is about 0.4 wt % to about 16 wt %.

The second mixture is then subjected to a photoisomerization reaction by passing the second mixture through a continuous flow photoisomerization reactor. The continuous flow photoisomerization reactor includes an ultraviolet (“UV”) light source that optionally can be surrounded by a filter. The reactor also comprises an input reservoir which can hold the starting reactant mixture (such as the second mixture of the method of the invention). A pump is used to pump the mixture from the reservoir, continuously (when in operation) through tubing connected to the reservoir. A portion of the tubing is wrapped around the UV light source, or alternatively, the filter surrounding the UV light source. The UV light source, filter and the wrapped portion of the tubing can be contained in an immersion vessel filled with fluid maintained at a constant temperature. Suitably, the temperature in the immersion vessel is maintained at a temperature of between about 10° C. to about 30° C., and more suitably at about 20° C. The flow rate of the mixture through the tubing may be a rate of about 2 mL/min to about 22 mL/min, however other flow rates may be used. In some embodiments, the rate of flow of the reaction mixture through the continuous flow photoisomerization reactor may be at least about 22 mL/min. After moving through the tubing the reaction mixture can flow into an output reservoir.

Passing through the continuous flow photoisomerization reactor the second mixture forms a third mixture which comprises a compound of formula II:

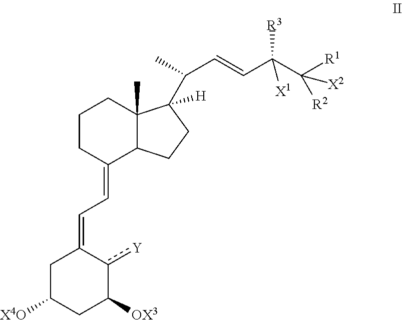

wherein R¹ and R² are identical or different and are hydrogen, hydroxyl, lower alkyl, lower fluoroalkyl, O-lower alkyl, lower alkenyl, lower fluoroalkenyl, O-lower alkenyl, O-lower acyl, O-aromatic acyl, lower cycloalkyl with the proviso that R¹ and R² cannot both be an alkenyl group, or taken together with the carbon to which they are bonded, form a C₃-C₈ cyclocarbon ring; R³ is lower alkyl, lower alkenyl, lower fluoroalkyl, lower fluoroalkenyl, O-lower alkyl, O-lower alkenyl, O-lower acyl, O-aromatic acyl or lower cycloalkyl; X¹ is hydrogen or hydroxyl, or, taken with R³, constitutes a bond when R³ is an alkenyl group, and X² is hydrogen or hydroxyl, or, taken with R¹ or R², constitutes a double bond, and X³ and X⁴ is hydrogen or tert-butyldimethylsilyl; and Y is a methylene group if the bond to Y is a double bond or is a methyl group or hydrogen if the bond to Y is a single bond. Optionally, the sensitizer can be removed from the third mixture by active carbon filtration, by chromatography, or by using a hydrazine-functionalized resin filter. If X³ and X⁴ is tert-butyldimethylsilyl, the compound of formula II is then mixed with a deprotection reagent to obtain the vitamin D₂ analog. In some embodiments, the deprotection reagent can include tetrabutylammonium fluoride.

In another embodiment the method above is performed where formula I comprises a compound represented by formula III:

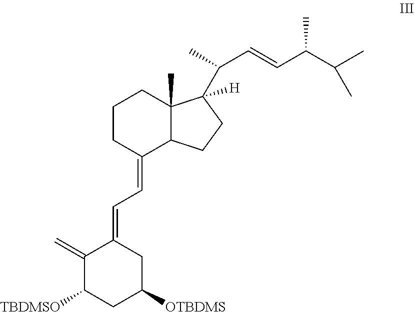 [SMILES: C=C1/C(=C/C=C2\CCC[C@@]3(C)C2CC[C@@H]3[C@H](C)/C=C/[C@H](C)C(C)C)C[C@@H](O[Si](C)(C)C(C)(C)C)C[C@@H]1O[Si](C)(C)C(C)(C)C]

and formula II comprises a compound of formula IV:

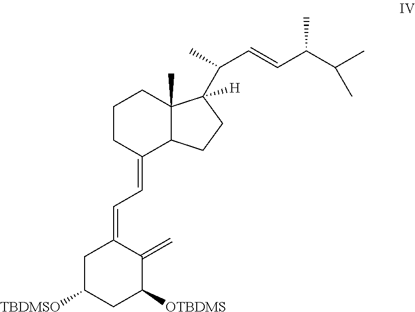 [SMILES: C=C1/C(=C\C=C2/CCC[C@@]3(C)C2CC[C@@H]3[C@H](C)/C=C/[C@H](C)C(C)C)C[C@@H](O[Si](C)(C)C(C)(C)C)C[C@@H]1O[Si](C)(C)C(C)(C)C]

with the resulting compound comprising doxercalciferol.

In yet another embodiment, the method above is performed where formula I comprises a compound represented by formula V:

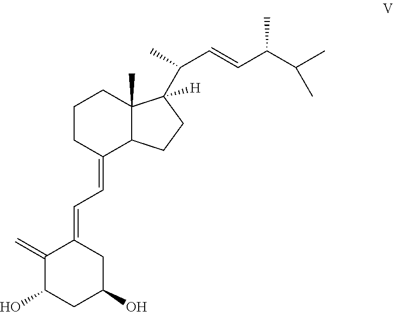 [SMILES: C=C1/C(=C/C=C2\CCC[C@@]3(C)C2CC[C@@H]3[C@H](C)/C=C/[C@H](C)C(C)C)C[C@@H](O)C[C@@H]1O]

### BRIEF DESCRIPTION OF THE DRAWINGS

FIG. 1 shows a diagram of a continuous flow photoisomerization reactor of the present application.

FIG. 2 shows a top perspective diagram of lamp assembly 106 of the continuous flow photoisomerization reactor of the present application.

### DETAILED DESCRIPTION

Before any embodiments of the present application are explained in detail, it is to be understood that the invention is not limited in its application to the details of construction and the arrangement of components set forth in the following description or illustrated in the following drawings. The invention is capable of other embodiments and of being practiced or of being carried out in various ways.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any nonclaimed element as essential to the practice of the invention.

As used herein, the term “lower” as a modifier for alkyl, alkenyl acyl, or cycloalkyl is meant to refer to a straight or branched, saturated or unsaturated hydrocarbon radical having 1 to 6 carbon atoms. Specific examples of such hydrocarbon radicals are methyl, ethyl, propyl, isopropyl, butyl, isobutyl, t-butyl, ethenyl, propenyl, butenyl, isobutenyl, isopropenyl, formyl, acetyl, propionyl, butyryl or cyclopropyl. The term “aromatic acyl” is meant to refer to a unsubstituted or substituted benzoyl group. The terms “O-lower alkyl”, “O-lower alkenyl”, “O-lower acyl” and “O-aromatic acyl” refers to lower alkyls, lower alkenyls, lower acyls and aromatic acyls respectively that have at least one oxygen atom. The term “lower fluoroalkenyl” refers to a straight or branched C₂₋₆ alkenyl group such as, for example, vinyl, allyl, isopropenyl, propenyl, butenyl, pentenyl, hexenyl, etc., having at least one fluorine atom.

The solvents used in the methods of the present invention can suitably include aliphatic hydrocarbons, cyclic hydrocarbons, aromatic hydrocarbons, alcohols, halogenated alkanes, ethers, esters, and mixtures thereof. In some embodiments, the solvent may be selected from the group consisting of heptane, methanol, toluene, 1,2-dichloroethane, t-butyl methyl ether, ethyl acetate, and mixtures thereof.

The sensitizers used in the methods of the present invention can suitably include an aromatic ketone, such as, for example, acetophenone, 1′-acetonaphthone, 2′-acetonaphthone, anisoin, anthrone, 9-acetylanthracene, benzophenone, benzoin, benzoin methyl ether, benzoin ethyl ether, benzoin isopropyl ether, benzoin isobutyl ether, benzoin phenyl acetate, benzalacetone, benzanthrone, benzoylacetone, 4,4′-bis(dimethylamino)-benzophenone, butyrophenone, chalcone, p-chloroacetophenone, alpha-chloroacetophenone, p-chlorobenzophenone, 2-chlorothioxanthone, desyl chloride, dibenzyl ketone, 2,2-diethoxyacetophenone, dibenzosuberone, dibenzalacetone, 4-dimethylaminob enzophenone, desoxyanisoin, desoxybenzoin, p-dimethylamino acetophenone, 2,5-dimethylbenzophenone, di-o-tolylketone, flavanone, flavone, 9-fluorenone, 4′-methoxypropiophenone, propiophenone, alpha-tetralone, thioxanthone, undecanophenone, valerophenone, xanthone, and mixtures thereof.

The deprotection reagents used in the methods of the present invention can suitably include hydrochloric acid, hydrochloric acid in ethanol, tetrabutylammonium fluoride (“TBAF”), a carboxylic acid chloride (e.g., acetyl chloride), thionyl chloride, oxalyl chloride, or a sulfonyl chloride (e.g., tosyl chloride). In some embodiments the deprotection reagent can comprise tetrabutylammonium fluoride.

As used herein, the term “crude purity” refers to the area under the curve of an HPLC chromatogram for the principal analyte.

The present application provides a novel method for generation of a vitamin D compound using a continuous flow photoisomerization reactor (e.g., a flow reactor). A method for removal of a sensitizer from the photolytic reaction mixture is also disclosed herein.

The chemical introduction of the C(1) hydroxyl group to a precursor, such as, for example, ergocalciferol (Scheme 1, structure 1), requires protection or isomerization of the triene moiety and often results in generation of the E-isomer of doxercalciferol, a vitamin D₂ analog, as the sole major constituent. The present application provides a method of producing the Z-isomer of doxercalciferol (Scheme 1, structure 2).

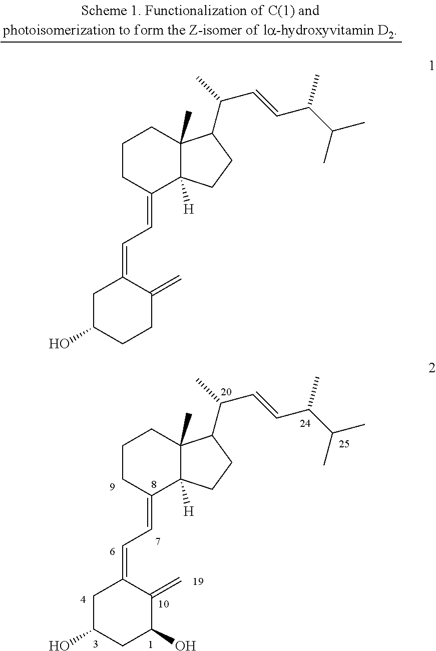 [SMILES: C=C1CC[C@H](O)C/C1=C/C=C1\CCC[C@]2(C)C([C@H](C)/C=C/[C@H](C)C(C)C)CC[C@@H]12 ; C=C1/C(=C\C=C2/CCC[C@]3(C)C([C@H](C)/C=C/[C@H](C)C(C)C)CC[C@@H]23)C[C@@H](O)C[C@@H]1O]

A process of preparing a vitamin D₂ analog is outlined in Scheme 2. Referring to Scheme 2, a compound represented by formula I may be combined with a solvent in a first step to form a first mixture.

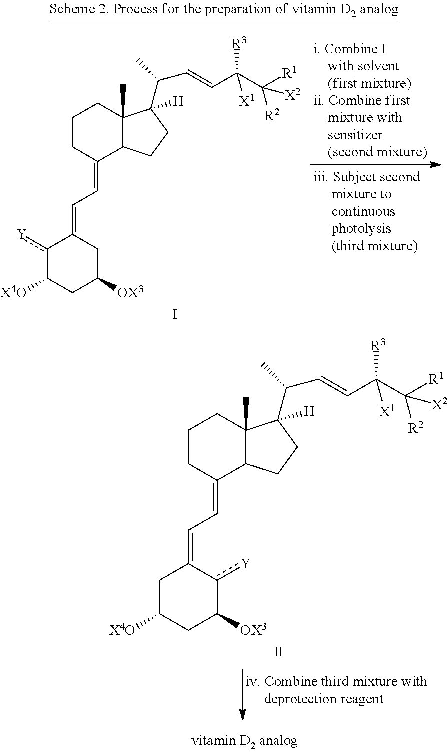

In some embodiments, the compound represented by formula I can include R¹ and R² that may be identical or different, and can be, for example, hydrogen, hydroxyl, lower alkyl, lower fluoroalkyl, O-lower alkyl, lower alkenyl, lower fluoroalkenyl, O-lower alkenyl, O-lower acyl, O-aromatic acyl, or lower cycloalkyl, with the proviso that R¹ and R² cannot both be an alkenyl group, or taken together with the carbon to which they are bonded, form a C₃-C₈ cyclocarbon ring. R³ may be, for example, lower alkyl, lower alkenyl, lower fluoroalkyl, lower fluoroalkenyl, O-lower alkyl, O-lower alkenyl, O-lower acyl, O-aromatic acyl, or lower cycloalkyl. X¹ may be hydrogen or hydroxyl, or, taken with R³, may constitute a bond when R³ is an alkenyl group, and X² is hydrogen or hydroxyl, or, taken with R¹ or R², may constitute a double bond, and X³ and X⁴ can be hydrogen or tert-butyldimethylsilyl. Y can be a methylene group if the bond to Y is a double bond or can be a methyl group or hydrogen if the bond to Y is a single bond.

In some embodiments of the present application, the compound represented by formula I may comprise a compound represented by formula III:

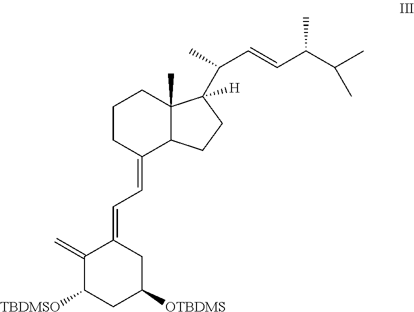 [SMILES: C=C1/C(=C/C=C2\CCC[C@@]3(C)C2CC[C@@H]3[C@H](C)/C=C/[C@H](C)C(C)C)C[C@@H](O[Si](C)(C)C(C)(C)C)C[C@@H]1O[Si](C)(C)C(C)(C)C]

where TBDMS=tert-butyldimethylsilyl.

In other embodiments of the present application, the compound represented by formula I may comprise a compound represented by formula V:

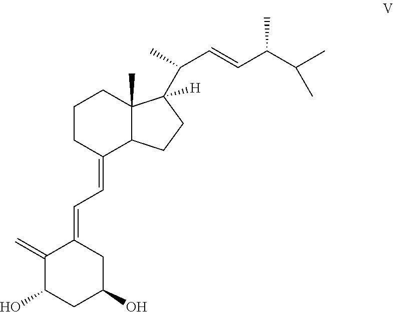 [SMILES: C=C1/C(=C/C=C2\CCC[C@@]3(C)C2CC[C@@H]3[C@H](C)/C=C/[C@H](C)C(C)C)C[C@@H](O)C[C@@H]1O]

In some embodiments, the concentration of the compound of formula I in the solvent (i.e., the first mixture) can be about 5 mg/mL to about 50 mg/mL. In some embodiments, the concentration of the compound of formula I in the solvent may be at least about 50 mg/mL.

Referring to Scheme 2, the first mixture can be combined with a sensitizer in a second step. In some embodiments, the ratio of the sensitizer to the first mixture suitably may be about 0.4 wt % to about 16 wt %, and more suitably may be 4 wt %.

Referring to Scheme 2, after the first mixture has been combined with a sensitizer to form the second mixture, the second mixture may be exposed to an ultraviolet (“UV”) light source in a third step to yield a third mixture, where the third mixture includes a compound represented by formula II. In some embodiments, the compound represented by formula II can include R¹ and R² that may be identical or different, and can be, for example, hydrogen, hydroxyl, lower alkyl, lower fluoroalkyl, O-lower alkyl, lower alkenyl, lower fluoroalkenyl, O-lower alkenyl, O-lower acyl, O-aromatic acyl, or lower cycloalkyl, with the proviso that R¹ and R² cannot both be an alkenyl group, or taken together with the carbon to which they are bonded, form a C₃-C₈ cyclocarbon ring. R³ may be, for example, lower alkyl, lower alkenyl, lower fluoroalkyl, lower fluoroalkenyl, O-lower alkyl, O-lower alkenyl, O-lower acyl, O-aromatic acyl, or lower cycloalkyl. X¹ may be hydrogen or hydroxyl, or, taken with R³, may constitute a bond when R³ is an alkenyl group, and X² is hydrogen or hydroxyl, or, taken with R¹ or R², may constitute a double bond, and X³ and X⁴ can be hydrogen or tert-butyldimethylsilyl. Y can be a methylene group if the bond to Y is a double bond or can be a methyl group or hydrogen if the bond to Y is a single bond.

In some embodiments of the present application, the compound represented by formula II may comprise a compound represented by formula IV:

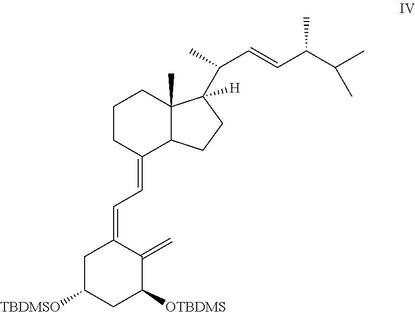 [SMILES: C=C1/C(=C\C=C2/CCC[C@@]3(C)C2CC[C@@H]3[C@H](C)/C=C/[C@H](C)C(C)C)C[C@@H](O[Si](C)(C)C(C)(C)C)C[C@@H]1O[Si](C)(C)C(C)(C)C]

where TBDMS=tert-butyldimethylsilyl.

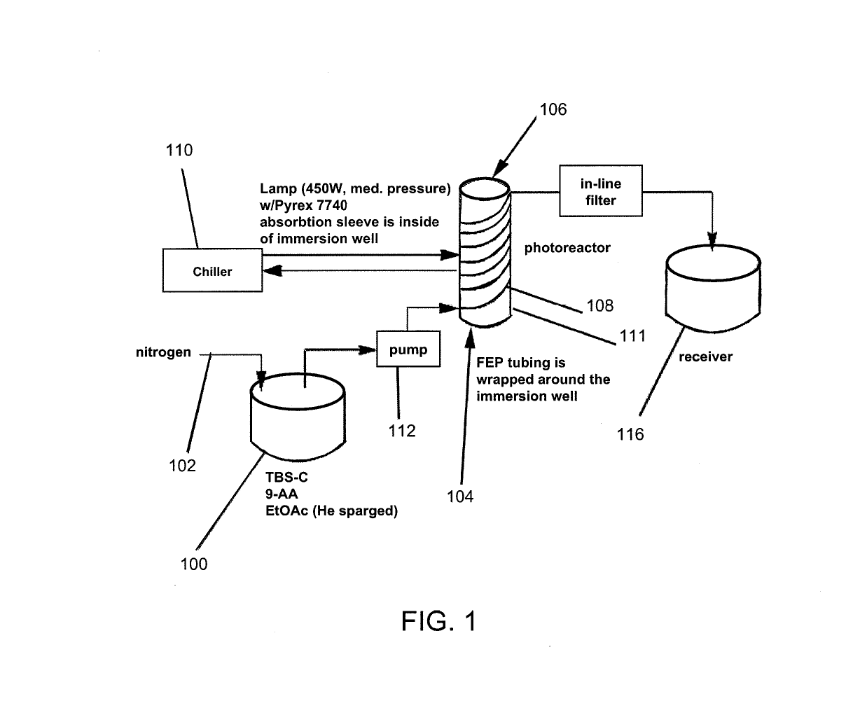

Exposure of mixtures of the present application to UV light may be accomplished through the use of a continuous flow photoisomerization reactor as shown in FIG. 1. Referring to FIG. 1, the continuous flow photoisomerization reactor may include an input reservoir 100 for containing a reaction mixture connected to a dry nitrogen stream 102. A photolysis assembly 104 can include a lamp assembly 106 and tubing 108 encircling the lamp assembly 106. The lamp assembly 106 can include a cylindrical UV lamp 105 surrounded by a sleeve 109 (e.g., a Pyrex or uranium glass filter) that can sit inside a glass cooling jacket 111. The cooling jacket 111 can be attached to a re-circulating chiller 110 to maintain the desired reaction temperature, which may suitably be between about 10° C. to about 30° C., and more suitably at about 20° C. In some embodiments, the tubing 108 may comprise chemically resistant fluoropolymer fluorinated ethylene propylene (“FEP”) tubing, but any suitable tubing would suffice. In some embodiments, a double layer of tubing 108 may be wrapped around the lamp assembly 106. A metal foil can jacket the photolysis assembly 104.

A pump 112 (e.g., an HPLC pump) can be used to transfer the reaction mixture from the input reservoir 100 through the tubing 108 of the photolysis assembly 104 at the desired rate. In some embodiments, the rate of flow of the reaction mixture through the continuous flow photoisomerization reactor may be a rate of about 2 mL/min to about 22 mL/min, however other flow rates may be used. In some embodiments, the rate of flow of the reaction mixture through the continuous flow photoisomerization reactor may be at least about 22 mL/min.

After passing through the photolysis assembly 104 the reaction mixture can flow into an output reservoir 116, which in some embodiments can be rotary evaporator flask, such as, for example, a 20 L rotary evaporation flask. In some embodiments, the reaction mixture may pass through an inline filter 114 before flowing into the output reservoir 116. The photolysis process can be carried out continuously and the product stream may be periodically or continuously analyzed for quality control. A real-time control could also expedite cycle time since the collected material could be evaporated as it was produced.

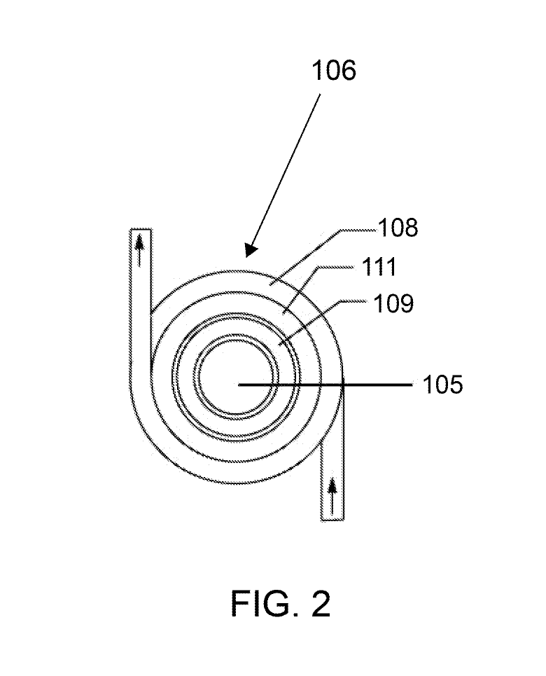

FIG. 2 shows a photograph of a continuous flow photoisomerization reactor assembled as described above.

Referring to Scheme 2, if X³ and X⁴ of the compound of formula II is tert-butyldimethylsilyl, then the compound is then mixed with a deprotection reagent to obtain the vitamin D₂ analog. In some embodiments, the deprotection reagent can include tetrabutylammonium fluoride.

In some embodiments of the present application, the sensitizer may be removed from the third mixture before the third mixture is combined with the deprotection reagent. Removal of the sensitizer may be accomplished, for example, by active carbon filtration, by chromatography, or by using a hydrazine-functionalized resin filter that can reversibly bind the 9-acetylanthracene sensitizer, making it removable by filtration (Scheme 3).

In some embodiments, the hydrazine-functionalized resin and the photosensitizer can be recovered and reused.

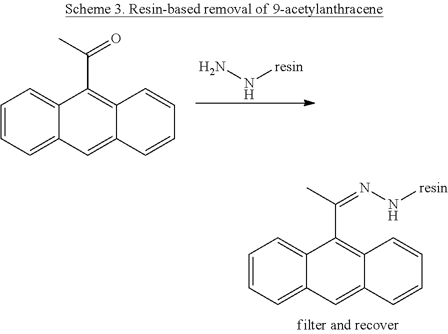

In some embodiments of the present application, the photolyzed solution may be directly eluted through a column of carbon (an in-line, continuous process) to remove the sensitizer. In one embodiment, the carbon column can be constructed by adding a layer of sand to a column followed by a 1/1 mixture of carbon/Celite (to increase flow rate), followed by another layer of sand. Commonly, a weight equivalent of carbon versus a weight equivalent of starting material may be used. The filtered solution can be concentrated to the desired volume and the solution can be used in the deprotection step directly.

In some embodiments, the vitamin D₂ analog product may desirably comprise doxercalciferol. In some embodiments, the doxercalciferol may include the Z-isomer of doxercalciferol (Scheme 1, structure 2). In some embodiments, the D₂ analog product may include the Z-isomer of doxercalciferol with at least about 80% to about 92% crude purity.

A process for preparing doxercalciferol is outlined in Scheme 4.

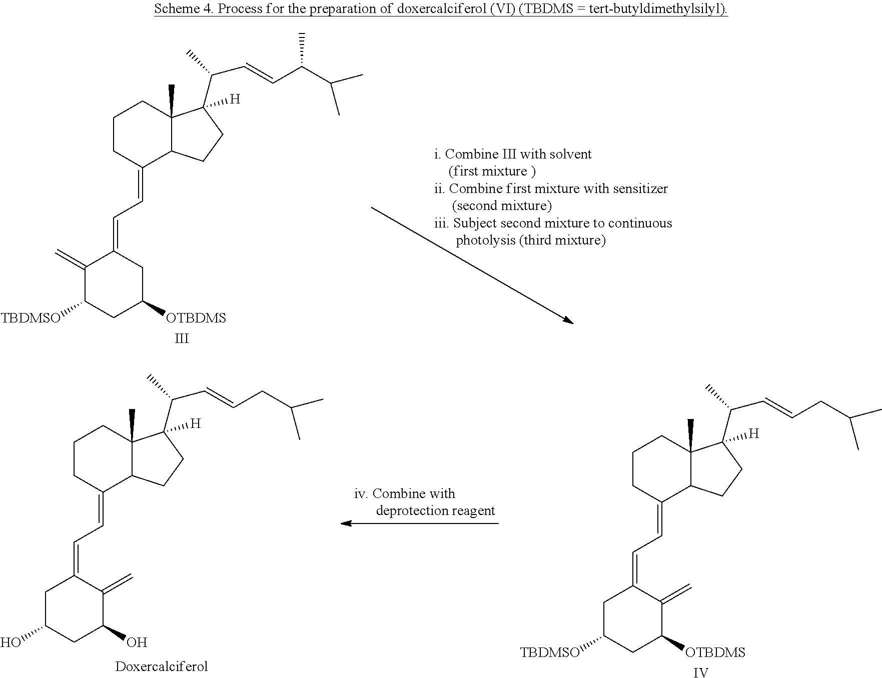

Referring to Scheme 4, a compound represented by formula III may be combined with a suitable solvent in step i), which suitably may comprise heptane. The first mixture can be combined with a sensitizer in step ii), which suitably may comprise 9-acetylanthracene. After the first mixture has been combined with a sensitizer to form the second mixture, the second mixture is exposed to a continuous photolysis by a continuous flow photoisomerization reactor of the present invention to yield a third mixture, where the third mixture includes a compound represented by formula IV. As described above, in some embodiments of the present application, the sensitizer may be removed from the third mixture after photolysis.

### EXAMPLE 1

### Set Up of Continuous Flow Photoisomerization Reactor

Two 100 mL flasks are provided as an input and output reservoirs. A photoreactor is provided which includes a 450 Watt Ultraviolet medium pressure mercury lamp (Hanovia lamp obtained from Ace Glass, Inc, www.aceglass.com, Cat. No. 7825-35) surrounded by a Pyrex® sleeve (obtained from Ace Glass, Inc, Cat. No. 7835-44), the UV lamp and Pyrex® sleeve being situated within a cooling jacket, such as an immersion well (obtained from Ace Glass, Inc, www.aceglass.com, Cat. No. 7874-35 or 7874-38). A power supply (obtained from Ace Glass, Inc, www.aceglass.com, Cat. No. 7830-60) is used to power the photoreactor. 50 ft of FEP tubing was obtained from Saint-Gobain Performance Plastics, AXI00002 Tube, FEP, 0.125″ (00)×(0.062″) 10, 50 ft; VWR (cat 63014-692). The FEP tubing is used to connect the input reservoir to a pump, and additional tubing is used to operatively connect the input reservoir to a portion of tubing which wraps around the outer surface of the cooling jacket of the photoreactor. The other end of the tubing is operatively connected to the output reservoir, such that there can be a continuous flow of fluid in the connected tubing from the input reservoir, through the tubing wrapped around the outer surface of the cooling jacket of the photoreactor to the output reservoir.

### EXAMPLE 2

### Doxercalciferol Preparation Process

A 100 mL flask was charged with the compound of formula III (0.567 g, 0.884 mmol), 9-acetylanthracene (21.4 mg, 0.097 mmol), and helium-sparged ethyl acetate (40 mL). The solution was pumped through the photoreactor at 20° C. and 14 mL/min. The photoreactor lamp was a 450 W, medium pressure mercury lamp (Hanovia) with a Pyrex absorption sleeve. After exiting the photoreactor, the solution passed through a column of Celite/activated carbon (2/1, w/w, 1.7 g) into a receiving flask. In-process HPLC analysis showed the compound of formula IV with 91% area purity. The solution was concentrated to 10 mL and a solution of acetyl chloride in ethanol (0.5 mL, 9%, v/v, 0.633 mmol) was added. The mixture was stirred at ambient temperature for 5.7 hours. HPLC analysis showed 96.5% conversion to the compound of formula VI. Solid sodium bicarbonate (2.0 g) and activated carbon (1.0 g) were added and the mixture was stirred at ambient temperature for 20 minutes. The mixture was filtered and the filtrate was concentrated. Methyl formate (50 mL) was added to the residue and the mixture was heated to boiling (˜40° C.). The volume was reduced by vacuum distillation at ambient temperature to 10 mL. The resulting slurry was cooled at −20° C. for 45 minutes, and then filtered. The flask and solids were washed with cold methyl formate and the solid was dried on the funnel to give the Z-isomer of doxercalciferol as a white solid (0.117 g, 32% recovery from III, 98.6% area purity).

Although the present disclosure has been described with several embodiments, myriad changes, variations, alterations, transformations, and modifications may be suggested to one skilled in the art, and it is intended that the present disclosure encompass such changes, variations, alterations, transformations, and modifications as they fall within the scope of the appended claims.

What is claimed is:

1. A method of making a vitamin D₂ analog, the method comprising:
a) mixing a compound represented by formula I: 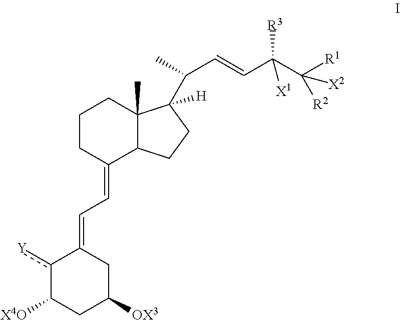
wherein R¹ and R² are identical or different and are hydrogen, hydroxyl, lower alkyl, lower fluoroalkyl, O-lower alkyl, lower alkenyl, lower fluoroalkenyl, O-lower alkenyl, O-lower acyl, O-aromatic acyl, lower cycloalkyl with the proviso that R¹ and R² cannot both be an alkenyl group, or taken together with the carbon to which they are bonded, form a C₃-C₈ cyclocarbon ring; R³ is lower alkyl, lower alkenyl, lower fluoroalkyl, lower fluoroalkenyl, O-lower alkyl, O-lower alkenyl, O-lower acyl, O-aromatic acyl or lower cycloalkyl; X¹ is hydrogen or hydroxyl, or, taken with R³, constitutes a bond when R³ is an alkenyl group, and X² is hydrogen or hydroxyl, or, taken with R¹ or R², constitutes a double bond, and X³ and X⁴ is hydrogen or tert-butyldimethylsilyl; and Y is a methylene group if the bond to Y is a double bond or is a methyl group or hydrogen if the bond to Y is a single bond, with a solvent to form a first mixture;
b) combining the first mixture with a sensitizer to form a second mixture;
c) subjecting the second mixture to a photoisomerization reaction by passing the second mixture through a continuous flow photoisomerization reactor to form a third mixture which comprises a compound of formula II: 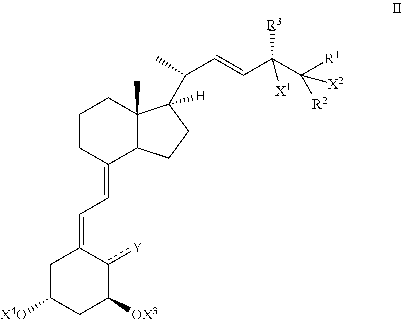
wherein R¹ and R² are identical or different and are hydrogen, hydroxyl, lower alkyl, lower fluoroalkyl, O-lower alkyl, lower alkenyl, lower fluoroalkenyl, O-lower alkenyl, O-lower acyl, O-aromatic acyl, lower cycloalkyl with the proviso that R¹ and R² cannot both be an alkenyl group, or taken together with the carbon to which they are bonded, form a C₃-C₈ cyclocarbon ring; R³ is lower alkyl, lower alkenyl, lower fluoroalkyl, lower fluoroalkenyl, O-lower alkyl, O-lower alkenyl, O-lower acyl, O-aromatic acyl or lower cycloalkyl; X¹ is hydrogen or hydroxyl, or, taken with R³, constitutes a bond when R³ is an alkenyl group, and X² is hydrogen or hydroxyl, or, taken with R¹ or R², constitutes a double bond, and X³ and X⁴ is hydrogen or tert-butyldimethylsilyl; and Y is a methylene group if the bond to Y is a double bond or is a methyl group or hydrogen if the bond to Y is a single bond; and
wherein if X³ and X⁴ of formula II is tert-butyldimethylsilyl, then formula II is mixed with a deprotection reagent to obtain the vitamin D₂ analog.

2. The method of claim 1 wherein the sensitizer comprises 9-acetylanthracene.

3. The method of claim 1 wherein the deprotection reagent comprises tetrabutylammonium fluoride.

4. The method of claim 1 wherein the deprotection reagent comprises hydrochloric acid.

5. The method of claim 1 wherein the solvent in step a) is selected from the group consisting of heptane, methanol, toluene, 1,2-dichloroethane, t-butyl methyl ether, ethyl acetate, and mixtures thereof.

6. The method of claim 5 wherein the solvent is deoxygenated.

7. The method of claim 6 wherein the solvent is deoxygenated by He sparging.

8. The method of claim 1 wherein the concentration of the compound of formula I in the solvent is about 5 mg/mL to about 50 mg/mL.

9. The method of claim 1 wherein the concentration of the compound of formula I in the solvent is at least about 50 mg/mL.

10. The method of claim 1 wherein the second mixture of step c) flows through the continuous flow photoisomerization reactor at a rate of about 2 mL/min to about 22 mL/min.

11. The method of claim 1 wherein the second mixture of step c) flows through the continuous flow photoisomerization reactor at a rate of at least about 22 mL/min.

12. The method of claim 1 wherein the second mixture of step c) flows through the continuous flow photoisomerization reactor at a temperature of between about 10° C. and about 30° C.

13. The method of claim 1 wherein the ratio of the sensitizer to the first mixture in step b) is about 0.4 wt % to about 16 wt %.

14. The method of claim 2 wherein the solvent in step a) comprises heptane, the sensitizer comprises 9-acetylanthracene, and the deprotection reagent comprises tetrabutylammonium fluoride.

15. The method of claim 2 wherein the solvent in step a) comprises heptane, the sensitizer comprises 9-acetylanthracene, and the deprotection reagent comprises hydrochloric acid.

16. The method of claim 1 further comprising a step after step c) wherein the sensitizer is removed from the third mixture by active carbon filtration, by chromatography, or by using a hydrazine-functionalized resin filter.

17. A method of making doxercalciferol, the method comprising:
a) mixing a compound represented by formula III: 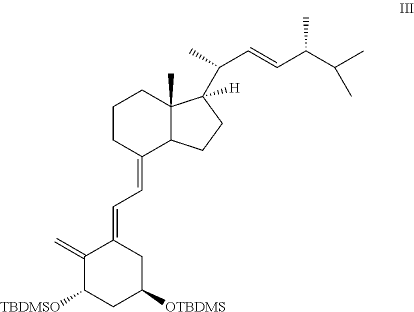 [SMILES: C=C1/C(=C/C=C2\CCC[C@@]3(C)C2CC[C@@H]3[C@H](C)/C=C/[C@H](C)C(C)C)C[C@@H](O[Si](C)(C)C(C)(C)C)C[C@@H]1O[Si](C)(C)C(C)(C)C]
with a solvent to form a first mixture;
b) combining the first mixture with a sensitizer to form a second mixture;
c) subjecting the second mixture to a photoisomerization reaction by passing the second mixture through a continuous flow photoisomerization reactor to form a third mixture which comprises a compound of formula IV: 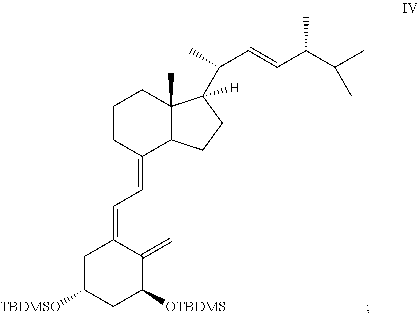 [SMILES: C=C1/C(=C\C=C2/CCC[C@@]3(C)C2CC[C@@H]3[C@H](C)/C=C/[C@H](C)C(C)C)C[C@@H](O[Si](C)(C)C(C)(C)C)C[C@@H]1O[Si](C)(C)C(C)(C)C]
and
d) mixing the compound of formula of IV with a deprotection reagent to obtain doxercalciferol.

18. The method of claim 17 wherein the sensitizer comprises 9-acetylanthracene.

19. The method of claim 17 wherein the deprotection reagent comprises tetrabutylammonium fluoride.

20. The method of claim 17 wherein the deprotection reagent comprises hydrochloric acid.

21. The method of claim 17 wherein the solvent in step a) is selected from the group consisting of heptane, methanol, toluene, 1,2-dichloroethane, t-butyl methyl ether, ethyl acetate, and mixtures thereof.

22. The method of claim 21 wherein the solvent is deoxygenated.

23. The method of claim 22 wherein the solvent is deoxygenated by He sparging.

24. The method of claim 17 wherein the concentration of the compound of formula III in the solvent is about 5 mg/mL to about 50 mg/mL.

25. The method of claim 17 wherein the concentration of the compound of formula III in the solvent is at least about 50 mg/mL.

26. The method of claim 17 wherein the second mixture of step c) flows through the continuous flow photoisomerization reactor at a rate of about 2 mL/min to about 22 mL/min.

27. The method of claim 17 wherein the second mixture of step c) flows through the continuous flow photoisomerization reactor at a rate of at least about 22 mL/min.

28. The method of claim 17 wherein the second mixture of step c) flows through the continuous flow photoisomerization reactor at a temperature of between about 10° C. and about 30° C.

29. The method of claim 17 wherein the ratio of the sensitizer to the first mixture in step b) is about 0.4 wt % to about 16 wt %.

30. The method of claim 17 wherein the solvent in step a) comprises heptane, the sensitizer comprises 9-acetylanthracene, and the deprotection reagent comprises tetrabutylammonium fluoride.

31. The method of claim 17 wherein the solvent in step a) comprises heptane, the sensitizer comprises 9-acetylanthracene, and the deprotection reagent comprises hydrochloric acid.

32. The method of claim 17 further comprising a step after step c) wherein the sensitizer is removed from the third mixture by active carbon filtration, by chromatography, or by using a hydrazine functionalized resin filter.

33. The method of claim 17 wherein the continuous flow photoisomerization reactor comprises an input reservoir which contains the second mixture, a pump which pumps the second mixture through tubing which surrounds a UV lamp, the tubing exiting into an output reservoir which collects the third mixture.

34. A method of making doxercalciferol, the method comprising:
a) mixing a compound represented by formula V with a solvent to form a first mixture; 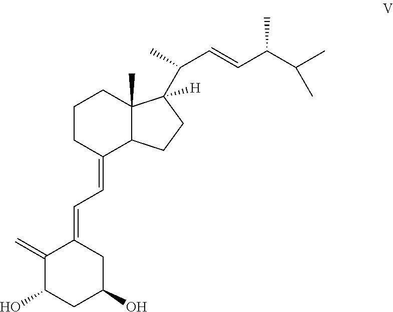 [SMILES: C=C1/C(=C/C=C2\CCC[C@@]3(C)C2CC[C@@H]3[C@H](C)/C=C/[C@H](C)C(C)C)C[C@@H](O)C[C@@H]1O]
b) combining the first mixture with a sensitizer to form a second mixture; and
c) subjecting the second mixture to a photoisomerization reaction by passing the second mixture through a continuous flow photoisomerization reactor to form doxercalciferol.

35. The method of claim 34 wherein the sensitizer comprises 9-acetylanthracene.

36. The method of claim 34 wherein the deprotection reagent comprises tetrabutylammonium fluoride.

37. The method of claim 34 wherein the deprotection reagent comprises hydrochloric acid.

38. The method of claim 34 wherein the solvent in step a) is selected from the group consisting of heptane, methanol, toluene, 1,2-dicholroethane, t-butyl methyl ether, ethyl acetate, and mixtures thereof.

39. The method of claim 38 wherein the solvent is deoxygenated.

40. The method of claim 39 wherein the solvent is deoxygenated by He sparging.

41. The method of claim 34 wherein the concentration of the compound of formula V in the solvent is about 5 mg/mL to about 50 mg/mL.

42. The method of claim 34 wherein the concentration of the compound of formula V in the solvent is at least about 50 mg/mL.

43. The method of claim 34 wherein the second mixture of step c) flows through the continuous flow photoisomerization reactor at a rate of about 2 mL/min to about 22 mL/min.

44. The method of claim 34 wherein the second mixture of step c) flows through the continuous flow photoisomerization reactor at a rate of at least about 22 mL/min.

45. The method of claim 34 wherein the second mixture of step c) flows through the continuous flow photoisomerization reactor at a temperature of between about 10° C. and about 30° C.

46. The method of claim 34 wherein the ratio of the sensitizer to the first mixture in step c) is about 0.4 wt % to about 16 wt %.

47. The method of claim 34 wherein the solvent in step a) comprises heptane, the sensitizer comprises 9-acetylanthracene, and the deprotection reagent comprises tetrabutylammonium fluoride.

48. The method of claim 34 further comprising a step after step c) wherein the sensitizer is removed from the doxercalciferol by active carbon filtration, by chromatography or by using a hydrazine functionalized resin filter.
